(19) United States
(12) Patent Application Publication
(10) Pub. No.: US 2009/0275957 A1
(43) Pub. Date: Nov. 5, 2009

(54) CLIP AND DELIVERY ASSEMBLY USED IN FORMING A TISSUE FOLD

(76) Inventors: Jason L. Harris, Mason, OH (US); Michael J. Stokes, Cincinnati, OH (US); Mark S. Zeiner, Mason, OH (US); Lawrence Crainich, Charlestown, NH (US)

Correspondence Address: WELSH & FLAXMAN LLC, 2000 DUKE STREET, SUITE 100, ALEXANDRIA, VA, 22314, US

(21) Appl. No.: 12/113,666

(22) Filed: May 1, 2008

(51) Int. Cl. A61B 17/128; A61B 17/122; A61B 17/064; A61B 17/068

(52) U.S. Cl. 606/142; 606/151; 606/219; 227/176.1

(57) ABSTRACT

A system for tissue apposition includes a delivery assembly having a tissue clip selectively secured thereto for the deployment and application of the tissue clip. The tissue clip is shaped and dimensioned for engaging spaced locations along a span of tissue and upon rotation thereof drawing the spaced locations together into apposition to thereby create a fold of tissue.

### BACKGROUND OF THE INVENTION

1. Field of the Invention

The invention relates to a method and apparatus for gastric reduction. More particularly, the invention relates to methods and apparatuses for creating folds along the exterior surface of the stomach.

2. Description of the Related Art

Obesity is a medical condition affecting more than 30% of the population in the United States. Obesity affects an individual's personal quality of life and contributes significantly to morbidity and mortality. Obese patients, i.e., individuals having a body mass index (“BMI”) greater than 30, often have a high risk of associated health problems (e.g., diabetes, hypertension and respiratory insufficiency), including early death. With this in mind, and as those skilled in the art will certainly appreciate, the monetary and physical costs associated with obesity are substantial. In fact, it is estimated the costs relating to obesity are in excess of 100 billion dollars in the United States alone. Studies have shown that conservative treatment with diet and exercise alone may be ineffective for reducing excess body weight in many patients. Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In a RYGB procedure a small stomach pouch is separated from the remainder of the gastric cavity and attached to a resectioned portion of the small intestine. This resectioned portion of the small intestine is connected between the “smaller” gastric cavity and a distal section of small intestine allowing the passage of food therebetween. The conventional RYGB procedure requires a great deal of operative time. Because of the degree of invasiveness, post-operative recovery can be quite lengthy and painful. Still more than 100,000 RYGB procedures are performed annually in the United States alone, costing significant health care dollars.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing a feeling of satiety. A band is placed around the stomach near the junction of the stomach and esophagus. The small upper stomach pouch is filled quickly, and slowly empties through the narrow outlet to produce the feeling of satiety. Other forms of bariatric surgery that have been developed to treat obesity include Fobi pouch, bilio-pancreatic diversion and gastroplasty or “stomach stapling”.

Morbid obesity is defined as being greater than 100 pounds over one's ideal body weight. For individuals in this category, gastric banding, RYGB or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss.

It is known to create cavity wall plications through endoscopic only procedures. However, operating solely within the interior of the gastric cavity limits the plication depth that can be achieved without cutting. Furthermore, access and visibility within the gastric and peritoneal cavities is limited in a purely endoscopic procedure as the extent of the reduction increases.

With the foregoing in mind, it is desirable to have a surgical weight loss procedure that is inexpensive, with few potential complications, and that provides patients with a weight loss benefit while buying time for the lifestyle changes necessary to maintain the weight loss. Further, it is desirable that the procedure be minimally invasive to the patient, allowing for a quick recovery and less scarring. The present invention provides such a procedure.

### SUMMARY OF THE INVENTION

It is, therefore, an object of the present invention to provide a system for tissue apposition including a delivery assembly having a tissue clip selectively secured thereto for the deployment and application of the tissue clip. The tissue clip is shaped and dimensioned for engaging spaced locations along a span of tissue and upon rotation thereof drawing the spaced locations together into apposition to thereby create a fold of tissue.

It is also an object of the present invention to provide a system for tissue apposition wherein the tissue clip includes an elongated body having a first end with a pointed free end, a second end with a pointed free end and a central section connecting the first end to the second end.

It is another object of the present invention to provide a system for tissue apposition wherein the first end includes a substantially U-shaped first hook member and the second end includes a substantially U-shaped second hook member.

It is a further object of the present invention to provide a system for tissue apposition wherein the central section includes a hinge portion.

It is also an object of the present invention to provide a system for tissue apposition wherein the delivery assembly includes an outer tube, a twirl mandrel and a clip retaining and straightening device, wherein the twirl mandrel and clip retaining and straightening device are shaped and dimensioned to move within the outer tube.

It is another object of the present invention to provide a system for tissue apposition wherein the tissue clip is an S-shaped hook tissue clip.

It is a further object of the present invention to provide a system for tissue apposition wherein the tissue clip includes first and second arms respectively defining first and second hooks.

It is also an object of the present invention to provide a system for tissue apposition wherein the first arm includes a pointed tip and the second arm includes a pointed tip.

It is another object of the present invention to provide a system for tissue apposition wherein the delivery assembly includes a twist lever and a clamp rod.

It is a further object of the present invention to provide a method for forming a fold of tissue including the steps of engaging a span of tissue at two spaced locations with a tissue clip, rotating the tissue clip to draw the spaced locations into apposition in a manner creating a tissue fold and securing the tissue in the folded configuration with adjacent surfaces of the tissue in contact.

It is also an object of the present invention to provide a method for forming a fold of tissue wherein the tissue clip includes an elongated body having a first end with a pointed free end, a second end with a pointed free end and a central section connecting the first end to the second end.

It is another object of the present invention to provide a method for forming a fold of tissue wherein the step of rotating includes rotating the central section to draw the first end closer to the second end.

It is a further object of the present invention to provide a method for forming a fold of tissue wherein the first end includes a substantially U-shaped first hook member and the second end includes a substantially U-shaped second hook member.

It is also an object of the present invention to provide a method for forming a fold of tissue wherein the central section includes a hinge portion.

It is another object of the present invention to provide a method for forming a fold of tissue wherein the tissue clip is an S-shaped hook tissue clip.

It is a further object of the present invention to provide a method for forming a fold of tissue wherein the tissue clip includes first and second arms respectively defining first and second hooks.

It is also an object of the present invention to provide a method for forming a fold of tissue wherein the step of rotating includes rotating the tissue clip to draw tissue snagged by the first hook and the second hook into apposition.

It is another object of the present invention to provide a method for forming a fold of tissue wherein the first arm includes a pointed tip and the second arm includes a pointed tip.

It is a further object of the present invention to provide a method for forming a fold of tissue further including the step of deploying the tissue clip within the body, wherein the tissue clip is maintained in a folded configuration during deployment, and the step of deployment includes straightening the tissue clip.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a view of the stomach with a fold formed therein in accordance with the present invention.

FIG. 2 is a cross sectional view showing a tissue fold formed with a clip in accordance with the present invention.

FIG. 3 is a perspective view of the clip shown with reference to FIG. 2 in its straightened configuration.

FIG. 4 is a side view of the clip shown in FIG. 2 in its coiled configuration.

FIG. 5 is a side view of the clip shown in FIG. 2 clip in its folded configuration for deployment.

FIGS. 6, 7 and 8 are cross sectional views of the process for applying the clip of FIG. 2.

FIG. 9 is a perspective view of the distal end of a twirl mandrel in accordance with the present invention.

FIG. 10 is a perspective view of the distal end of a clip retaining and straightening device in accordance with the present invention.

FIG. 11 is a perspective view of the distal end of an outer tube in accordance with the present invention.

FIG. 12 is a perspective view of an S-shaped hook tissue clip in accordance with an alternate embodiment.

FIG. 13 is a perspective view of the S-shaped hook tissue clip of FIG. 12 secured to a deployment delivery assembly.

FIG. 14 is a side view of the distal end of the twist lever shown in FIG. 13.

FIGS. 15 and 16 show use of the S-shaped hook tissue clip.

FIG. 17 is a perspective view of an S-shaped hook tissue clip in accordance with a further embodiment of the present invention.

FIG. 18 is a perspective view of the S-shaped hook tissue clip of FIG. 17 secured to a deployment delivery assembly.

FIG. 19 is a side view of the distal end of the twist lever shown in FIG. 18.

FIGS. 20 and 21 show use of the S-shaped hook tissue clip.

### DESCRIPTION OF THE PREFERRED EMBODIMENTS

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as a basis for teaching one skilled in the art how to make and/or use the invention.

With reference to the various embodiments described below, the present invention provides a system for tissue apposition. The system includes a delivery assembly having a tissue clip selectively secured thereto for the deployment and application of the tissue clip. The tissue clip is shaped and dimensioned for engaging spaced locations along a span of tissue and upon rotation thereof drawing the spaced locations together into apposition to thereby create a fold of tissue. In practice, the system is implemented by engaging a span of tissue at two spaced locations with a tissue clip, rotating the tissue clip to draw the spaced locations into apposition in a manner creating a tissue fold, and securing the tissue in the folded configuration with adjacent surfaces of the tissue in contact.

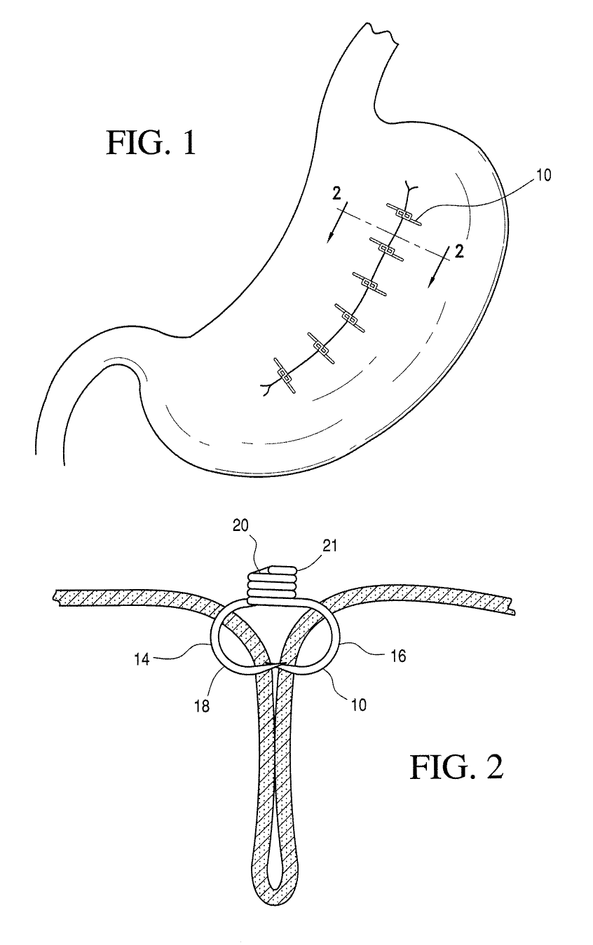

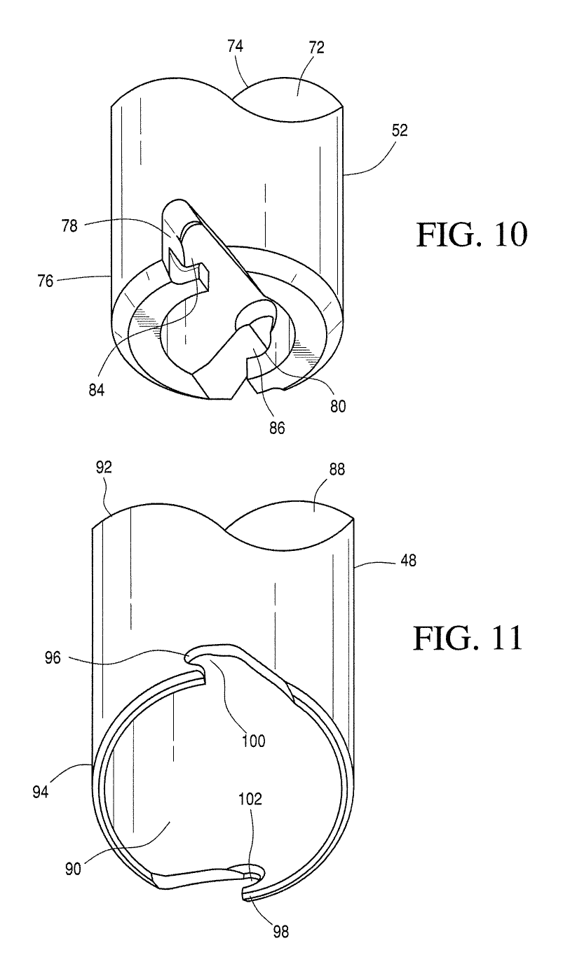

With reference to FIGS. 1 to 11, a tissue clip 10 and delivery assembly 12 are disclosed. The tissue clip 10 and delivery assembly 12 are designed for engaging spaced locations along a span of the external surface of the stomach, that is, they are used in snagging stomach tissue in two locations (for example, and in accordance with a preferred embodiment of the present invention, approximately 38 mm apart), and gathering the snagged portions of the stomach to approximate the tissue and fix the outer surfaces of the stomach in a manner creating a tissue fold. The procedure may be repeated numerous times along a line of tissue to create a long internal fold within the body, for example, on the serosal surface of the stomach to create a fold reducing the internal capacity of the stomach. After the tissue is drawn into apposition creating a fold in accordance with the present invention, the apposed tissue will over time bond together ultimately negating the need for permanent fasteners to hold the tissue in a folded configuration.

Although a preferred spacing of approximately 38 mm is disclosed above for the purpose of disclosing the present invention, it is contemplated the specific spacing may vary greatly based upon the application of the present invention. In particular, although a tissue clip 10 used in accordance with the present invention and described below in greater detail will have a set length, the width of the actual tissue bite (that is, the extent or span of tissue between the engagement and penetration points for the first hook member 22 and second hook member 34 of the tissue clip 10) will vary depending upon the characteristics of the tissue and the manner in which the tissue clip 10 is applied. In addition, although a particular tissue fold arrangement is disclosed herein, it is contemplated a variety of tissue fold patterns may be applied to impair gastric motility, provide inlet/outlet restrictions and/or to otherwise change the rate of gastric emptying. Examples of tissue fold patterns are described in further detail in commonly owned U.S. patent application Ser. No. 11/779,322, filed on Jul. 18, 2007, the contents of which are incorporated herein by reference in its entirety. In this way, the reduction or alteration in stomach volume obtains satiety with less food consumption and consequently weight reduction results.

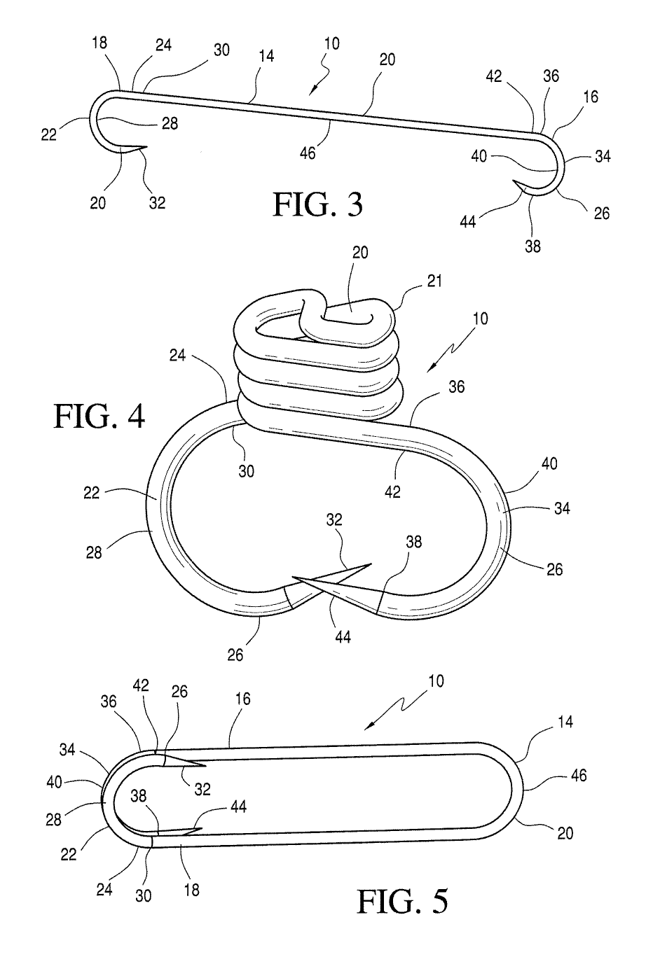

More particularly, and with reference to FIGS. 3, 4 and 5, the tissue clip 10 includes an elongated body 14 having a first end 16, a second end 18 and a central section 20 connecting the first end 16 to the second end 18. The first end 16 includes a substantially U-shaped first hook member 22 having a first leg 24 and a second leg 26 with a connecting member 28 therebetween. The end 30 of the first leg 24 is connected to the central section 20 of the elongated body 14 and the second leg 26 includes a pointed free end 32 shaped and dimensioned for partial or complete penetration and engagement of tissue. The second end 18 of the tissue clip 10 similarly includes a substantially U-shaped second hook member 34 having a first leg 36 and a second leg 38 with a connecting member 40 therebetween. The end 42 of the first leg 36 is connected to the central section 20 of the elongated body 14 and the second leg 38 includes a pointed free end 44 shaped and dimensioned for partial or complete penetration and engagement of tissue.

The second legs 26, 38 of the respective first end 16 and second end 18 face each other in an opposed relationship and are preferably aligned along substantially the same longitudinal axis; that is, the pointed free ends 32, 44, extend toward the respective opposite ends of the tissue clip 10.

As such, the tissue clip 10 is shaped and dimensioned such that the pointed free ends 32, 44 of the respective first end 16 and second end 18 may engage spaced tissue sections along an expanse of tissue. As will be explained below in greater detail, once the pointed free ends 32, 44 of the respective first end 16 and second end 18 are seated within or through tissue sections along the expanse of the tissue, the central section 20 is twisted (or rotated). Twisting of the central section 20 effectively shortens the length of the central section 20 and draws the first end 16 closer to the second end 18, and consequently draws the tissue section in which the pointed free end 32 of the first end 16 is seated closer to the tissue section in which the pointed free end 44 of the second end 18 is seated.

In practice, and as will be explained in greater detail as the procedure associated with the present tissue clip 10 is described, the central section 20 of the tissue clip 10 is folded along a mid-region 46 thereof to reduce the profile of the tissue clip 10 and allow for ease of passage into the body cavity where it may be applied to tissue in a desired manner. In accordance with a preferred embodiment of the present invention, the central section 20 is composed of the same material as the remainder of the tissue clip. However, the central section 20, and, in particular, the mid-region 46 of the elongated body 14 is preferably a metal that is heat treated, work hardened or constructed with differing geometries to achieve the stiffness and bending characteristics desired in accordance with the present invention. In accordance with a preferred embodiment of the present invention, the central section 20 of the elongated body 14 is formed with a rectangular configuration allowing for bending in the manner described herein, but providing for substantial rigidity for preventing bending in a transverse direction.

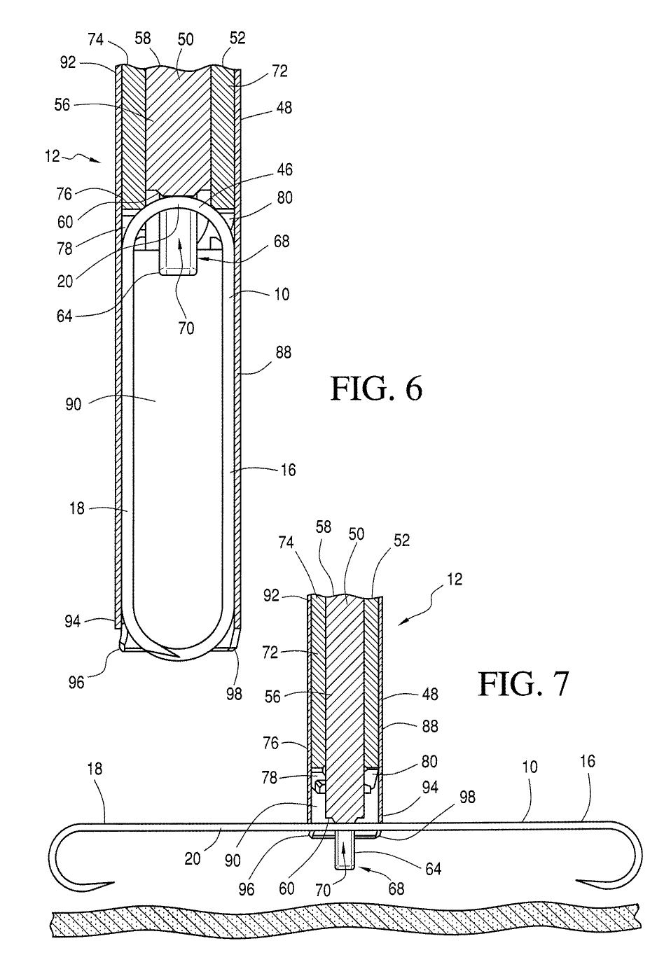

Referring to FIGS. 6 to 11, delivery of the tissue clip 10 to a desired location is achieved by the provision of a delivery assembly 12 specifically adapted for delivering the tissue clip 10 to a specific site in a low profile configuration as shown in FIG. 5, expanding the tissue clip 10 into a configuration as shown in FIG. 3 for secure tissue engagement, and twisting (or rotating) the central section 20 of the elongated body 14 of the tissue clip 10 to draw tissue into apposition to create a tissue fold as desired in accordance with the present invention. The delivery assembly 12 includes an outer tube 48 shaped and dimensioned for housing the various components of the delivery assembly 12, as well as the tissue clip 10 in its lower profile folded configuration. In an exemplary embodiment, the outer diameter of outer tube 48 is sized for passage through a small (3-5 mm) trocar although larger diameters are also within the scope of this invention. The moving components of the delivery assembly 12 include a twirl mandrel 50 which moves within a clip retaining and straightening device 52, both of which move within outer tube 48.

In general and as described below in greater detail with reference to FIGS. 1, 2, 6 and 8, the tissue clip 10 is deployed in the following manner. Once the delivery assembly 12 is properly positioned, relative linear motion between the twirl mandrel 50 and clip retaining and straightening device 52 straightens the tissue clip 10 (preferably the clip retaining and straightening device 52 when retracted pushes the tissue clip 10 against the twirl mandrel 50). The outer tube 48 then engages the straightened tissue clip 10 and the tissue clip engages the tissue (this step can occur at any point after tissue clip 10 straightening, this location is preferred as tissue clip 10 has maximum engagement with device). The clip retaining and straightening device 52 disengages the tissue clip 10 and moves proximally to allow space for twirling the central section 20 of the elongated body 14 of the tissue clip 10 within outer tube 48. The twirl mandrel 50 is then used to twirl the central section 20 of the elongated body 14 of the tissue clip 10. Finally, the outer tube 48 is disengaged from tissue clip 10.

More particularly, and with reference to FIGS. 6, 7, 8 and 9, the twirl mandrel 50 is shaped and dimensioned for engaging the central section 20 of the elongated body 14 of the tissue clip 10 and permitting twisting of the central section 20 in a manner reducing the effective length of the elongated body 14. The twirl mandrel 50 includes an elongated body 56 having a first end 58 and a second end 60. The first end 58 includes a handle (not shown) for actuation by a medical practitioner performing the specific procedure. The second end 60 includes a first prong 64 and a second prong 66 extending therefrom along the longitudinal axis of the twirl mandrel 50 and the first and second prongs 64, 66 are substantially parallel and have a recess 70 therebetween. As such, the first and second prongs 64, 66 are spaced apart a predetermined distance (thereby forming a “fork”) allowing for placement of the central section 20 of the tissue clip 10 within the recess 70 between the first and second prongs 64, 66 to allow for manipulation of the central section 20 of the tissue clip 10. In accordance with a preferred embodiment, the prongs 64, 66 and the central section 20 are dimensioned to facilitate engagement with other components (that is, the clip retaining and straightening device 52 and the outer tube 48) only when the opening 68 of the recess 70 is aligned with the central section 20 so as to prevent undesirable rotation prior to twirling in accordance with the present invention.

The twirl mandrel 50 is shaped and dimensioned to fit within the clip retaining and straightening device 52 for manipulation of the tissue clip 10. Referring to FIGS. 6, 7, 8 and 10, the clip retaining and straightening device 52 generally includes a tubular body 72 shaped and dimensioned for retaining the tissue clip 10 and straightening or expanding the tissue clip 10 prior to tissue engagement (snagging) and apposition (caused by twirling of the central section 20 of the elongated body 14). The clip retaining and straightening device 52 includes a first end 74 and a second end 76. As with the twirl mandrel 50, the first end 74 of the clip retaining and straightening device 52 is adapted for engagement by a medical practitioner during performance of a procedure in accordance with the present invention. The second end 76 of the clip retaining and straightening device 52 includes opposed first and second hooks 78, 80 which hold the tissue clip 10 in place during insertion of the tissue clip 10 into the outer tube 48. The respective first and second hooks 78, 80 include a radially oriented recess 84, 86 shaped and dimensioned for receiving and retaining the central section 20 of the tissue clip 10 during performance of a procedure in accordance with the present invention. Briefly, and as discussed below in greater detail, the first and second hooks 78, 80 are also used to straighten the tissue clip 10 by pulling proximally against the twirl mandrel 50. The first and second hooks 78, 80 release the tissue clip 10 by slight rotation in a predetermined direction, for example, clockwise in accordance with a preferred embodiment of the present invention. The clip retaining and straightening device 52 is retracted for twirling but advancing it distally, after twirling, strips the coil 21 of the tissue clip 10 from the first and second prongs 64, 66 of the twirl mandrel 50.

Referring to FIGS. 6, 7, 8 and 11, the outer tube 48 of the present invention is shown. The outer tube 48 includes a tubular body 88 having a central lumen 90. The outer tube 48 functions by containing the tissue clip 10 for insertion through the cannula (not shown). The outer tube 48 also guides the first and second ends 16, 18 of the tissue clip 10 as they are being drawn closer together. As with the twirl mandrel 50 and the clip retaining and straightening device 52, the outer tube 48 includes a first end 92 and a second end 94. The first end 92 of the outer tube 48 is adapted for engagement by a medical practitioner during performance of a procedure in accordance with the present invention. The second end 94 of the outer tube 48 includes opposed recesses 96, 98, defining first and second hooks 100, 102, at the distal end thereof. The recesses 96, 98 are shaped and dimensioned for holding spaced sections of the tissue clip 10 maintaining the orientation of the tissue clip 10 as the twirl mandrel 50 rotates and coils the central section 20 so that tissue is pulled inward and does not have the tendency to pull circumferentially. After completion of the twirling, the first and second hooks 100, 102 are released from the tissue clip 10 by slight rotation.

Referring to FIGS. 1, 2 and 6-8, the procedure for application of a tissue clip 10 in accordance with the present invention is disclosed. Referring to FIG. 6, a cross section of the components of the present delivery assembly 12 is shown. In the configuration shown with reference to FIG. 6, the delivery assembly 12 is ready for insertion through a cannula. The tissue clip 10 is in its folded low profile configuration in the crotch of the recess 70 formed by the first and second prongs 64, 66 of the twirl mandrel 50. The opposed first and second hooks 78, 80 of the clip retaining and straightening device 52 hold the tissue clip 10 in place during storage and insertion through the outer tube 48. The outer tube 48 contains the tissue clip's distal first and second ends 16, 18 and fits through the cannula.

Referring to FIG. 7, as the delivery assembly 12 is positioned for the tissue clip 10 to retain tissue, the tissue clip 10 remains within the recess 70 formed between the first and second prongs 64, 66 of the twirl mandrel 50. The outer tube 48 is then retracted such that the tissue clip 10 is exposed and ready for straightening. The clip retaining and straightening device 52 is then retracted causing the tissue clip 10 to straighten as the central section 20 is pulled against the twirl mandrel 50. The tissue clip 10 is then moved into engagement with the outer tube 48 as the first and second hooks 100, 102 are hooked onto the backspan, that is, the central section 20, of the tissue clip 10 and is ready for twirling of the central section 20 of the elongated body 14 of the in accordance with the present invention.

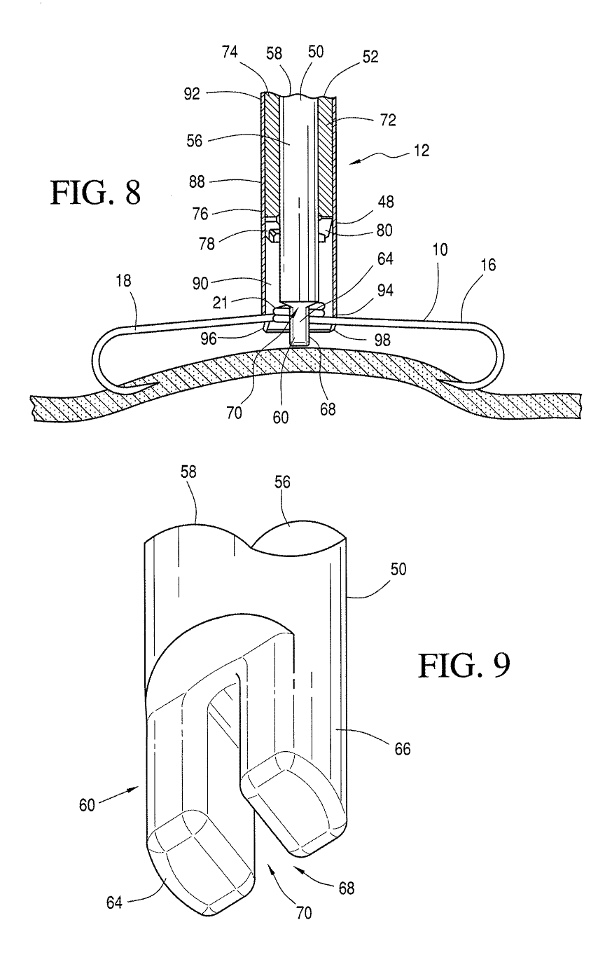

Referring to FIG. 8, the tissue clip 10 is finished being curled forming a coil 21 wherein the effective length of the central section 20 of the elongated body 14 reduced. Notice that the coil 21 that has been curled together allows the first and second ends 16, 18 of the tissue clip 10 to approximate tissue in accordance with the present invention. After completion of the twirling, the first and second hooks 100, 102 of the outer tube 48 are unhooked from the central section 20 of the tissue clip 10 and the clip retaining and straightening device 52 is moved distally to eject the coiled tissue clip 10 from within the recess 70.

More particularly, the present delivery assembly 12 operates in the following manner. The tissue clip 10 is folded about its mid-region 46 to fit into the outer tube 48 that fits through the cannula directing it to the gastric cavity. The tissue clip 10 is placed onto the first and second hooks 78, 80 of the clip retaining and straightening device 52. The twirl mandrel 50 is inserted into the clip retaining and straightening device 52 with the tissue clip 10 in the recess 70 between the first prong 64 and second prong 66 of the twirl mandrel 50. The outer tube 48 is slid over the entire clip 10 to house it for insertion.

The delivery assembly 12 is then inserted through a cannula of a trocar assembly. Once inside the body cavity and out of the distal end of the cannula, the outer tube 48 is retracted to expose the tissue clip 10. The clip retaining and straightening device 52 is moved proximally, causing the tissue clip 10 to open from its folded configuration with the central section 20 of the tissue clip 10 straightened out from the folded configuration in which it was previously held while within the outer tube 48. The central section 20 of the tissue clip 10 need not be absolutely straight or the first and second ends 16, 18 thereof exactly 180° apart. The outer tube 48 is then moved distally and rotated such that the first and second hooks 100, 102 of the outer tube 48 are latched onto the first and second ends 16, 18 of the tissue clip 10. The clip retaining and straightening device 52 is then rotated to release the tissue clip 10. Thereafter, the clip retaining and straightening device 52 is retracted. One pointed free end 32 of the tissue clip 10 is inserted into the tissue (that is, penetrates) of the stomach in a desired location. The other pointed free end 44 of the tissue clip 10 is inserted into the stomach tissue (that is, penetrates). The twirl mandrel 50 is rotated to twist and coil the central section 20 of the elongated body 14 and thereby gather tissue by pulling the first end 16 and the second end 18 of the tissue clip 10 together to desired approximation of the tissue. The first and second hooks 100, 102 of the outer tube 48 are unlatched by rotation of the outer tube 48 and the outer tube 48 is retracted to clear the tissue clip 10. The clip retaining and straightening device 52 is then advanced to strip the coiled tissue clip 10 off the twirl mandrel 50.

The present tissue clip 10 and delivery assembly 12 provide a tissue clip 10 that is stiff and resistant to deformation at the first and second ends 16, 18 where the free pointed ends 32, 44 are located and the central section 20 of the elongated body 14 is easily coiled by the twirl mandrel. Methods to provide strength to the first and second ends 16, 18 while minimizing material quantities include altering the material properties (e.g., hardening, etc.) in that portion of the clip and/or optimizing the cross sectional area of the clip in this region to resist deformation under the anticipated loads. One example of a simple geometry that would resist deformation is to make the cross sectional area rectangular in the clip with the longer edge of the rectangle corresponding to the plane of the first and second ends 16, 18 and the shorter edge of the rectangle being perpendicular to the plane of the first and second ends 16, 18. The central section 20 may similarly be easily coiled by the twirl mandrel through choices of material properties and geometry. Methods to provide ease of twisting in the central section 20 while minimizing material quantities include altering the material properties (e.g., annealing, etc.) in that portion of the tissue clip 10 and/or optimizing the cross sectional area of the tissue clip 10 in this region to facilitate bending under the anticipated loads. One example of a simple geometry that would facilitate bending is to make the cross sectional area rectangular in the central section 20 of the tissue clip 10 with the longer edge of the rectangle corresponding lying in a plane perpendicular to the plane in which the first and second hook members 22, 34 lie and the shorter edge of the rectangle lying in the same plane in which the first and second hook members 22, 34 lie. In this way, the clip may be rolled upon itself with the resulting size of the twirled central region being of a reduced size. The method of unfolding the tissue clip 10 to a deployed position allows for a low profile clip which is easy for insertion. The coiling of the central section 20 of the tissue clip 10 to bring the pointed free ends 32, 44 of the first and second ends 16, 18 of the tissue clip 10 together to approximate tissue allows for the creation of a fold as desired in accordance with gastric reduction procedures.

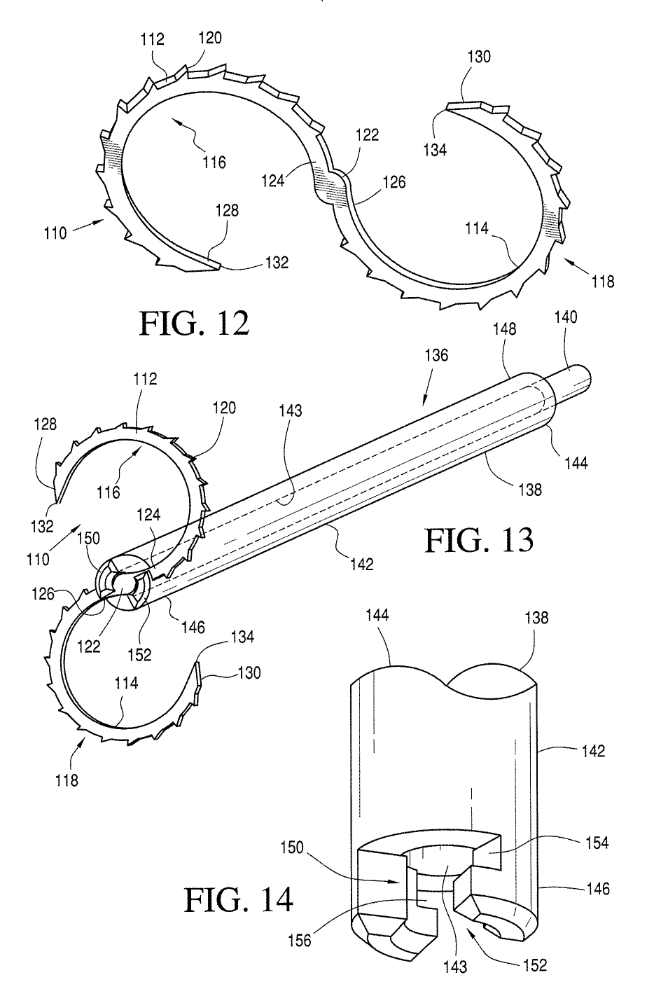

In accordance with an alternate embodiment, and with reference to FIGS. 12 to 16, the clip previously disclosed with reference to FIGS. 1 to 11, is replaced with a surgical S-shaped hook tissue clip 110. In particular, the hook shaped tissue clip 110 has first and second arms 112, 114 respectively defining first and second hooks 116, 118 in accordance with a preferred embodiment. The present tissue clip 110 allows for strength and sufficient permanency in the formation of the fold. The hooks 116, 118 of the tissue clip 110 snag stomach tissue, gather it together to reduce stomach volume and then lock the tissue folds together by rotation of the tissue clip 110 in the manner discussed below. In addition, barbs 120 formed upon the hooks 116, 118 of the tissue clip 110 assist in preventing reversal of the tissue clip 110 and release of the fold. Exemplary embodiments of this clip may be found in published PCT application WO2006037399, the entire contents of which are herein incorporated by reference.

More particularly, and with reference to FIGS. 12 and 13, a surgical S-shaped hook tissue clip 110 is disclosed. First and second arms 112, 114 extend from a center point 122 of the tissue clip 110. The center point 122 is designated as the point of rotation for the tissue clip 110 as will be appreciated based upon the following disclosure. Each of the first and second arms 112, 114 includes a first end 124, 126 which is coupled to the center point 122 of the tissue clip 110 and a second, or free end 128, 130. The free end 128, 130 of each of the respective first and second arms 112, 114 includes a pointed tip 132, 134 shaped and dimensioned for penetration within tissue as described herein in greater detail. The first and second arms 112, 114 are of a circular configuration and extend approximately ¾ of a complete circle. Starting from the point of rotation, each of the first and second arms 112, 114 has the same direction of rotation. Therefore, if one goes along a respective arm 112, 114 as far as its pointed tip 132, 134, one is moving along a right hand curve or in a clockwise direction.

Barbs 120 are provided on the outer sides of both the first and second arms 112, 114. In accordance with a preferred embodiment, the tissue clip 110 is substantially flat and the first and second arms 112, 114, therefore, lie in approximately the same plane. It is envisioned, however, that in alternate embodiments, the pointed tips 132, 134 need not be in the same plane as the center point 122.

To make it easier to grip the tissue clip 110 in the vicinity of the center point 122 thereof, for example, with the aid of a delivery assembly 136 as discussed below in greater detail, the area surrounding the center point 122 is of a flattened configuration (with surfaces parallel to one another extending perpendicular to the plane of the paper), while the first and second arms 112, 114 although shown as being flattened may be of a round configuration, for example, or are dimensioned more strongly in the plane of the paper than perpendicular thereto.

A delivery assembly 136 is provided for use in conjunction with the present S-shaped hook tissue clip 110. The delivery assembly 136 includes a twist lever 138 and a clamp rod 140. The twist lever 138 and clamp rod 140 are shaped and dimensioned for insertion through a cannula. As with the twirl mandrel of the prior embodiment, the twist lever 138 is shaped and dimensioned for engaging the center point 122 of the tissue clip 110 and permitting twisting thereof. The twist lever 138 includes an elongated body 142 having a central lumen 143, as well as a first end 144 and a second end 146. The first end 144 includes a handle 148 for actuation by a medical practitioner performing the specific procedure. The second end 146 includes a first prong 150 and a second prong 152. The first and second prongs 150, 152 extend along the longitudinal axis of the distal end of the twist lever 138 and the first and second prongs 150, 152 are substantially parallel. The first and second prongs 150, 152 include lateral recesses 154, 156 allowing the twist lever 138 to hook onto the S-shaped hook tissue clip 110 to hold it in preparation for twisting. To keep the S-shaped hook tissue clip 110 from falling off the twist lever 138, the clamp rod 140 fits within the twist lever 138 and is forced against the tissue clip 110 creating friction between the tissue clip 110 and the lateral recesses 154, 156 in which the tissue clip 110 sits during deployment and held in position by a mechanism in the handle 148.

In practice, and with reference to FIGS. 14, 15 and 16, the S-shaped hook tissue clip 110 is inserted into the body cavity, through a cannula, in the proximity of the stomach separate from the delivery assembly 136. The delivery assembly 136, that is, the twist lever 138 and clamp rod 140, are inserted through the cannula. The twist lever 138 picks up the S-shaped hook tissue clip 110 and manipulates it into the first and second prongs 150, 152 of the twist lever 138. The clamp rod 140 is pushed distally and clamped onto the S-shaped hook tissue clip 110 for securely holding it at the distal end of the twist lever 138. The device, with the S-shaped hook tissue clip 110, can now be manipulated to snag tissue as desired.

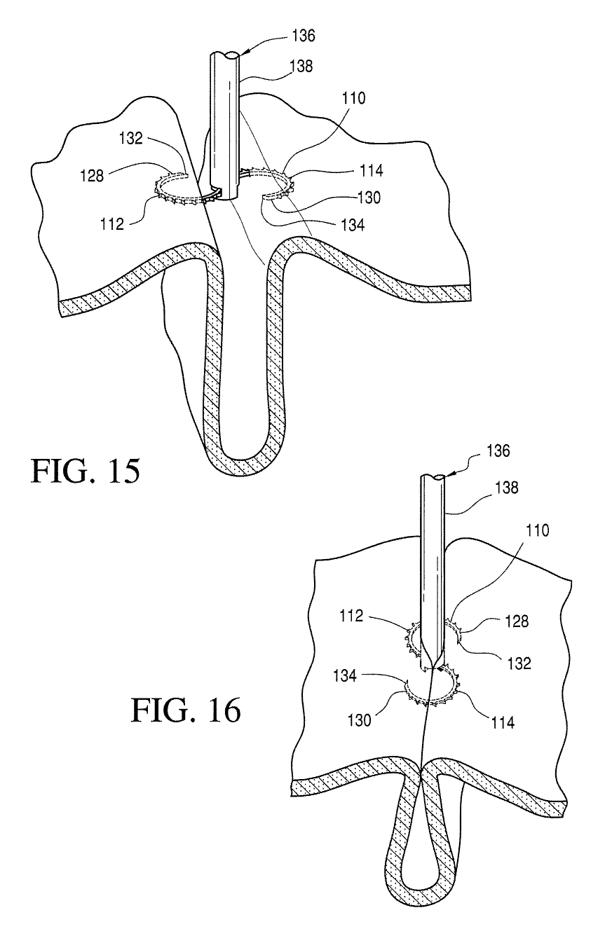

The process for engaging tissue and drawing it together is shown with reference to FIGS. 13, 14, 15 and 16. In the first step, the S-shaped hook tissue clip 110 is placed into the treatment area with the aid of the twist lever 138 which grips the S-shaped hook tissue clip 110 near the center point 122 of the tissue clip 110 and extends substantially perpendicular to the plane in which the S-shaped hook tissue clip 110 lies. The pointed tips 132, 134 touch and penetrate the area of the tissue apposition, respectively, and in the condition shown in FIG. 15, are ready for rotation to draw the tissue into apposition creating a fold.

The S-shaped hook tissue clip 110 is now turned clockwise. FIG. 15 shows the state after a 90° turn. As will be seen, the tissue margins have to follow the first and second arms 112, 114, for which reason they are moved toward one another. Referring now to FIG. 16, the tissue is shown after the S-shaped hook tissue clip 110 has been turned 180° as can be seen from the free ends 128, 130 of the S-shaped hook tissue clip 110. In this position the first and second arms 112, 114 penetrate both sides of the tissue in apposition. The tissue margins now touch and the tissue is drawn in apposition.

When the margins of tissue are approximated and folded, the delivery assembly 136 will twist the S-shaped hook tissue clip 110 to continue to gather tissue until it is firmly clamped. The barbs 120 on the S-shaped hook tissue clip 110 prevent reversal of the tissue clip 110. At this point, the clamp rod 140 is released, the twist lever 138 is counter rotated and the delivery assembly 136 is removed.

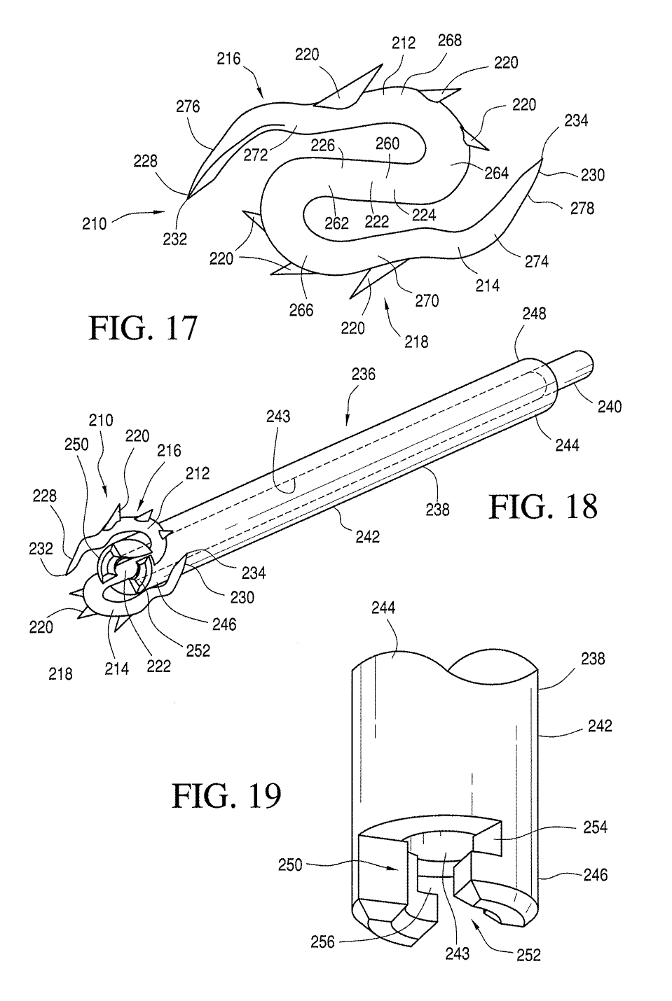

Referring to FIGS. 17-21, an alternate embodiment may employ a hook clip different from that disclosed above with reference to FIGS. 12 to 16. More particularly, the clip previously disclosed with reference to FIGS. 12 to 16, is replaced with a surgical S-shaped hook tissue clip 210. In particular, the S-shaped hook tissue clip 210 has first and second arms 212, 214 respectively defining first and second hooks 216, 218 in accordance with a preferred embodiment. The present tissue clip 210 allows for strength and sufficient permanency in the formation of the fold. The generally C-shaped hooks 216, 218 of the tissue clip 210 snag stomach tissue, gather it together to reduce stomach volume and then lock the tissue folds together by rotation of the tissue clip 210 in the manner discussed below. In addition, barbs 220 formed upon the hooks 216, 218 of the tissue clip 210 assist in preventing reversal of the tissue clip 210 and release of the fold. Also, the shape of these hooks 216, 218 more easily allows passage through a trocar while maximizing the tissue approximation strength of the tissue clip 210.

More particularly, and with reference to FIGS. 17 and 18, a surgical S-shaped hook tissue clip 210 is disclosed. First and second arms 212, 214 extend from a center point 222 of the tissue clip 210. The center point 222 is designated as the point of rotation for the tissue clip 210 as will be appreciated based upon the following disclosure. Each of the first and second arms 212, 214 includes a first end 224, 226 which is coupled to the center point 222 of the tissue clip 210 and a second, or free end 228, 230. The free ends 228, 230 of the respective first and second arms 212, 214 include pointed tips 232, 234 shaped and dimensioned for penetration within tissue as described herein in greater detail. The first and second arms 212, 214 are of a rectilinear configuration and each includes a first segment 260, 262, a first elbow 264, 266, a connecting segment 268, 270, a second elbow 272, 274 and a second segment 276, 278 which ends in a pointed tip extending in a direction substantially aligned with a circumference about which the hook tissue clip 210 rotates in accordance with the present invention and as discussed below in greater detail. Starting from the point of rotation, each of the first and second arms 212, 214 has the same direction of rotation. Therefore, if one goes along a respective arm 212, 214 as far as its pointed tip 232, 234, one is moving along a right hand curve or in a clockwise direction.

Barbs 220 are provided on the outer sides of both the first and second arms 212, 214. In accordance with a preferred embodiment, the tissue clip 210 is substantially flat and the first and second arms 212, 214, therefore, lie in approximately the same plane.

To make it easier to grip the tissue clip 210 in the vicinity of the center point 222 thereof, for example, with the aid of a delivery assembly 236 as discussed below in greater detail, the area surrounding the center point 222 is of a flattened configuration (with surfaces parallel to one another extending perpendicular to the plane of the paper), while the first and second arms 212, 214 are made of round material, for example, or are dimensioned more strongly in the plane of the paper than perpendicular thereto.

A delivery assembly 236 is provided for use in conjunction with the present S-shaped hook tissue clip 210. The delivery assembly 236 includes a twist lever 238 and a clamp rod 240. The twist lever 238 and clamp rod 240 are shaped and dimensioned for insertion through a cannula. As with the twirl mandrel of the prior embodiment, the twist lever 238 is shaped and dimensioned for engaging the center point 222 of the tissue clip 210 and permitting twisting thereof. The twist lever 238 includes an elongated body 242 having a central lumen 243, as well as a first end 244 and a second end 246. The first end 244 includes a handle 248 for actuation by a medical practitioner performing the specific procedure. The second end 246 includes a first prong 250 and a second prong 252. The first and second prongs 250, 252 extend along the longitudinal axis of the distal end of the twist lever 238 and the first and second prongs 250, 252 are substantially parallel. The first and second prongs 250, 252 include lateral recesses 254, 256 allowing the twist lever 238 to hook onto the S-shaped hook tissue clip 210 to hold it, in preparation for twisting. To keep the S-shaped hook tissue clip 210 from falling off the twist lever 238, the clamp rod 240 fits within the twist lever 238 and is forced against the tissue clip 210 creating friction between the tissue clip 210 and the lateral recesses 254, 265 in which the tissue clip 210 sits during deployment and held in position by a mechanism in the handle 248.

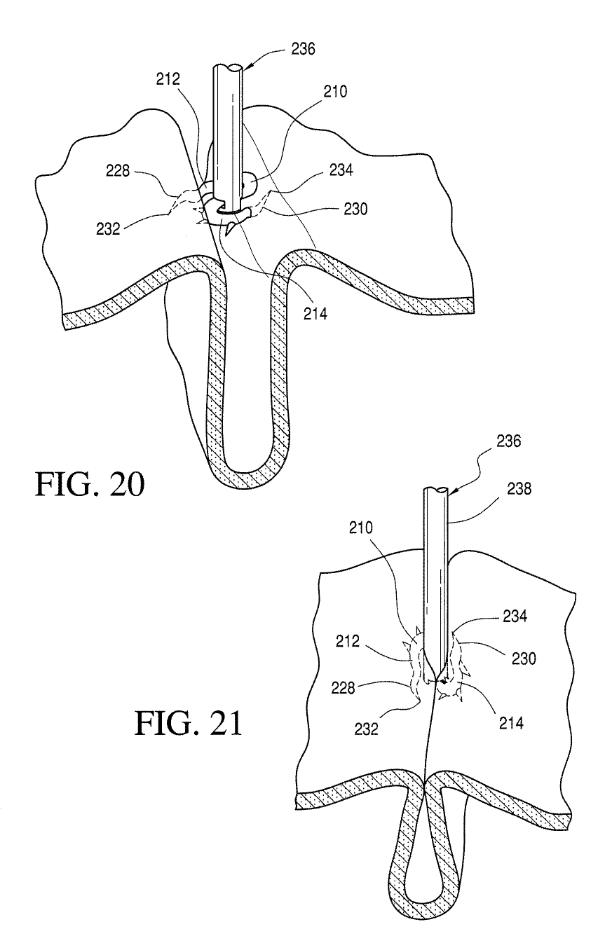

In practice, and with reference to FIGS. 20 and 21, the S-shaped hook tissue clip 210 is inserted into the body cavity, through a cannula, in the proximity of the stomach separate from the delivery assembly. The delivery assembly 236, that is, the twist lever 238 and clamp rod 240, are inserted through the cannula. The twist lever 238 picks up the S-shaped hook tissue clip 210 and manipulates it into the first and second prongs 250, 252 of the twist lever 238. The clamp rod 240 is pushed distally and clamped onto the S-shaped hook clip 210 for securely holding it at the distal end of the twist lever 238. The device, with the S-shaped hook tissue clip 210, can now be manipulated to snag tissue as desired.

The process for engaging tissue and drawing it together is shown with reference to FIGS. 20 and 21. In the first step, the S-shaped hook tissue clip 210 is placed into the treatment area with the aid of the twist lever 238 which grips the S-shaped hook tissue clip 210 near the center point 222 of the tissue clip 210 and extends substantially perpendicular to the plane in which the S-shaped hook tissue clip 210 lies. The pointed tips 132, 134 touch the area of the tissue apposition, respectively, and in the condition shown in FIG. 20, are ready to fully penetrate into the tissue in the area of the tissue apposition.

The S-shaped hook tissue clip 210 is now turned clockwise. FIG. 20 shows the state after a 90° rotation. As will be seen, the tissue margins have to follow the first and second arms 212, 214, for which reason they are moved toward one another. At this point, the long length of the second segment 276, 278 has dug into the tissue. Referring now to FIG. 21, the tissue is shown after the S-shaped hook tissue clip 210 has been rotated 180° as can be seen from the free ends 228, 230 of the S-shaped hook tissue clip 210. At this point, the first and second elbows 264, 266, 272, 274 are drawn through the tissue and the tissue margins now touch and the tissue is drawn in apposition.

When the margin of tissue are approximated, the delivery assembly 236 will twist the S-shaped hook tissue clip 210 to continue to gather tissue until it is firmly clamped. The barbs 220 on the S-shaped hook tissue clip 210 prevent reversal of the tissue clip 210. At this point, the clamp rod 240 is released, the twist lever 238 is counter rotated and the delivery assembly 236 is removed.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used system is obtained and if necessary cleaned. The system can then be sterilized. In one sterilization technique, the system is placed in a closed and sealed container, such as a plastic or TYVEK bag. The container and system are then placed in a field of radiation that can penetrate the container, such as gamma radiation, x-rays, or high-energy electrons. The radiation kills bacteria on the system and in the container. The sterilized system can then be stored in the sterile container. The sealed container keeps the system sterile until it is opened in the medical facility.

It is preferred that the device is sterilized. This can be done by any number of ways known to those skilled in the art including beta or gamma radiation, ethylene oxide, and/or steam.

While the preferred embodiments have been shown and described, it will be understood that there is no intent to limit the invention by such disclosure, but rather, is intended to cover all modifications and alternate constructions falling within the spirit and scope of the invention.

1. A system for tissue apposition, comprising:
a delivery assembly having a tissue clip selectively secured thereto for deployment and application of the tissue clip;
the tissue clip being shaped and dimensioned for engaging spaced locations along a span of the tissue and upon rotation thereof drawing the spaced locations together into apposition to thereby create a fold of the tissue.

2. The system according to claim 1, wherein the tissue clip includes an elongated body having a first end with a pointed free end, a second end with a pointed free end and a central section connecting the first end to the second end.

3. The system according to claim 2, wherein the first end includes a substantially U-shaped first hook member and the second end includes a substantially U-shaped second hook member.

4. The system according to claim 2, wherein the central section includes a hinge portion.

5. The system according to claim 2, wherein the delivery assembly includes an outer tube, a twirl mandrel and a clip retaining and straightening device, wherein the twirl mandrel and clip retaining and straightening device are shaped and dimensioned to move within the outer tube.

6. The system according to claim 1, wherein the tissue clip is an S-shaped hook tissue clip.

7. The system according to claim 6, wherein the tissue clip includes first and second arms respectively defining first and second hooks.

8. The system according to claim 7, wherein the first arm includes a pointed tip and the second arm includes a pointed tip.

9. The system according to claim 6, wherein the delivery assembly includes a twist lever and a clamp rod.

10. A method for forming a fold of tissue, comprising:
engaging a span of tissue at two spaced locations with a tissue clip;
rotating the tissue clip to draw the spaced locations into apposition in a manner creating a tissue fold;
securing the tissue in a folded configuration with adjacent surfaces of the tissue in contact.

11. The method according to claim 10, wherein the tissue clip includes an elongated body having a first end with a pointed free end, a second end with a pointed free end and a central section connecting the first end to the second end.

12. The method according to claim 11, wherein the step of rotating includes rotating the central section to draw the first end closer to the second end.

13. The method according to claim 12, wherein the first end includes a substantially U-shaped first hook member and the second end includes a substantially U-shaped second hook member.

14. The method according to claim 12, wherein the central section includes a hinge portion.

15. The method according to claim 10, wherein the tissue clip is an S-shaped hook tissue clip.

16. The method according to claim 15, wherein the tissue clip includes first and second arms respectively defining first and second hooks.

17. The method according to claim 16, wherein the step of rotating includes rotating the tissue clip to draw the tissue snagged by the first hook and the second hook into apposition.

18. The method according to claim 17, wherein the first arm includes a pointed tip and the second arm includes a pointed tip.

19. The method according to claim 10, further including the step of deploying the tissue clip within a body, wherein the tissue clip is maintained in the folded configuration during deployment, and the step of deployment includes straightening the tissue clip.
